(19) United States
(12) Patent Application Publication
(10) Pub. No.: US 2005/0033410 A1
(43) Pub. Date: Feb. 10, 2005

(54) Vascular prothesis having flexible configuration

(75) Inventors: Michael Hogendijk, Santa Clara, CA (US); Eric Leopold, Redwood City, CA (US); Miles Alexander, Fremont, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: NovoStent Corporation, Santa Clara, CA (US)

(21) Appl. No.: 10/911,435

(22) Filed: Aug. 4, 2004

Related U.S. Application Data
(63) Continuation-in-part of application No. 10/342,427, filed on Jan. 13, 2003.
(60) Provisional application No. 60/436,516, filed on Dec. 24, 2002.

(51) Int. Cl. A61F 2/06

(52) U.S. Cl. 623/1.15

(57) ABSTRACT

An implantable vascular prosthesis having improved flexibility is provided comprising a helical body portion having a reduced delivery configuration and an expanded deployed configuration, the helical body portion comprising a plurality of cells interconnected by hinge points that enhances flexibility of the vascular prosthesis in the reduced delivery configuration.

### REFERENCE TO RELATED APPLICATIONS

The present application is a continuation-in-part application of U.S. patent application Ser. No. 10/342,427, filed Jan. 13, 2003, which claims the benefit of the filing date of U.S. provisional patent application Ser. No. 60/436,516, filed Dec. 24, 2002.

### FIELD OF THE INVENTION

The present invention relates to an implantable vascular prosthesis configured for use in a wide range of applications, and more specifically, a vascular prosthesis having improved flexibility in a reduced delivery configuration.

### BACKGROUND OF THE INVENTION

Vascular stenting has become a practical method of reestablishing blood flow to a patient's diseased vasculature. Today there are a wide range of intravascular prostheses on the market for use in the treatment of aneurysms, stenoses, and other vascular irregularities. Balloon expandable and self-expanding stents are well known for restoring patency in a stenosed vessel, e.g., after an angioplasty procedure, and the use of coils and stents are known techniques for treating aneurysms.

Previously-known self-expanding stents generally are retained in a contracted delivery configuration using an outer sheath, and then self-expand when the sheath is retracted. Such stents commonly have several drawbacks, for example, the stents may experience large length changes during expansion (referred to as “foreshortening”) and may shift within the vessel prior to engaging the vessel wall, resulting in improper placement. Additionally, many self-expanding stents have relatively large delivery profiles because the configuration of their struts limits further compression of the stent. Accordingly, such stents may not be suitable for use in smaller vessels, such as cerebral vessels and coronary arteries.

For example, PCT Publication WO 00/62711 to Rivelli describes a stent comprising a helical mesh coil having a plurality of turns and including a lattice having a multiplicity of pores. The lattice is tapered along its length. In operation, the plurality of turns are wound into a reduced diameter helical shape, then constrained within a delivery sheath. The delivery sheath is retracted to expose the distal portion of the stent and anchor the distal end of the stent. As the delivery sheath is further retracted, the subsequent individual turns of the stent unwind to conform to the diameter of the vessel wall.

The stent described in the foregoing publication has several drawbacks. For example, due to friction between the turns and the sheath, the individual turns of the stent may bunch up, or overlap with one another, when the delivery sheath is retracted. In addition, once the sheath is fully retracted, the turns may shift within the vessel prior to engaging the vessel wall, resulting in improper placement of the stent. Moreover, the stent pattern may not be sufficiently flexible to allow the stent to be rolled to a small delivery profile diameter.

In view of these drawbacks of previously known devices, it would be desirable to provide apparatus and methods for an implantable vascular prosthesis comprising a stent that exhibits a high degree of flexibility in a reduced delivery profile.

It also would be desirable to provide apparatus and methods for a vascular prosthesis having a body portion featuring cells that do not engage each other when the body portion is rolled to a reduced delivery configuration.

It further would be desirable to provide apparatus and methods for a vascular prosthesis having cell configurations that provide substantially linear helical features throughout the pattern, wherein the linear features may include angular changes and/or hinge points to provide a vascular prosthesis having increased flexibility in the reduced delivery configuration and good radial strength.

### SUMMARY OF THE INVENTION

In view of the foregoing, it is an object of the present invention to provide apparatus and methods for an implantable vascular prosthesis comprising a stent that exhibits a high degree of flexibility in a reduced delivery profile.

It is also an object of the present invention to provide apparatus and methods for a vascular prosthesis having a body portion featuring cells that that do not engage each other when the body portion is rolled to a reduced delivery configuration.

It is another object of the present invention to provide apparatus and methods for a vascular prosthesis having cell configurations that provide substantially linear helical features throughout the pattern, wherein the linear features may include angular changes and/or hinge points.

It is a further object of the present invention to provide apparatus and methods for a vascular prosthesis that has a substantially small delivery configuration, thereby allowing the prosthesis to be used in smaller vessels.

These and other objects of the present invention are accomplished by providing an implantable vascular prosthesis having improved flexibility, comprising a helical body portion capable of assuming a reduced delivery configuration and an expanded deployed configuration. The helical body portion comprises a cell configuration that provides increased flexibility in the reduced delivery configuration. The cell configuration preferably comprises one or more substantially parallel struts that extend helically for the length of the helical body portion. More preferably, the cell configuration comprises a linear series of cells that are interconnected by hinged articulations.

The vascular prosthesis of the present invention may include cells having corners that define hinge elements, thereby allowing shape changes to occur in a planar fashion. Improved flexibility of the prosthesis in the reduced delivery configuration also may be achieved by providing a higher level of angularity among within the cell configuration. The radial force of the vascular prosthesis may be controlled by varying the placement of hinges within the cell configuration or by varying the angularity within the cell configuration. The radial force of the body portion also may be controlled by varying the angle that the cells are aligned along the longitudinal axis of the stent.

According to some embodiments, the individual cells that make up the body portion are sized so that the cell length is a fraction of the circumference of the body portion in the reduced delivery configuration. In other embodiments, the cells are dimensioned to provide a whole number of cells per reduced diameter circumference.

In a preferred embodiment, the vascular prosthesis comprises a shape memory material, such as a nickel-titanium alloy, and includes a distal anchor section coupled to a proximal helical body portion having a plurality of turns. The cell configuration of the proximal portion includes features aligned substantially parallel to the helix of the helical body portion, such as angular changes and/or hinge points. By providing a cell configuration having a greater number of angular changes and hinge points, a vascular prosthesis having greater flexibility may be obtained. Preferably, the cell configurations provide substantially linear helical components throughout the pattern.

Methods of using the vascular prosthesis of the present invention, for example also are provided.

### BRIEF DESCRIPTION OF THE DRAWINGS

Further features of the invention, its nature and various advantages will be more apparent from the accompanying drawings and the following detailed description of the preferred embodiments, in which:

FIG. 1 is a perspective view of a first embodiment of a vascular prosthesis constructed in accordance with the present invention;

FIGS. 2A-2B are, respectively, a perspective view of an alternative embodiment of a vascular prosthesis of the present invention and plan view of a portion of the unwound helical body portion;

FIG. 3 is a plan view of a portion of an unwound body portion of a further alternative embodiment of a vascular prosthesis of the present invention;

FIG. 4 is a side view, partly in section, of a vascular prosthesis of the present invention disposed within an illustrative delivery catheter; and

FIGS. 5A-5G are side-sectional views depicting use of the apparatus of FIG. 4 to perform angioplasty and to delivery a vascular prosthesis.

### DETAILED DESCRIPTION OF THE INVENTION

The present invention is directed to an implantable vascular prosthesis configured for use in a wide range of applications, such as treating aneurysms, maintaining patency in a vessel, and allowing for the controlled delivery of therapeutic agents to a vessel wall. The prosthesis has a helical configuration that provides a substantially smaller delivery profile than previously-known devices. In a preferred embodiment, the stent includes a helical body portion joined to a radially expandable distal portion. Importantly, however, the principles of the present invention may be advantageously applied to a stent comprising a helical body portion alone.

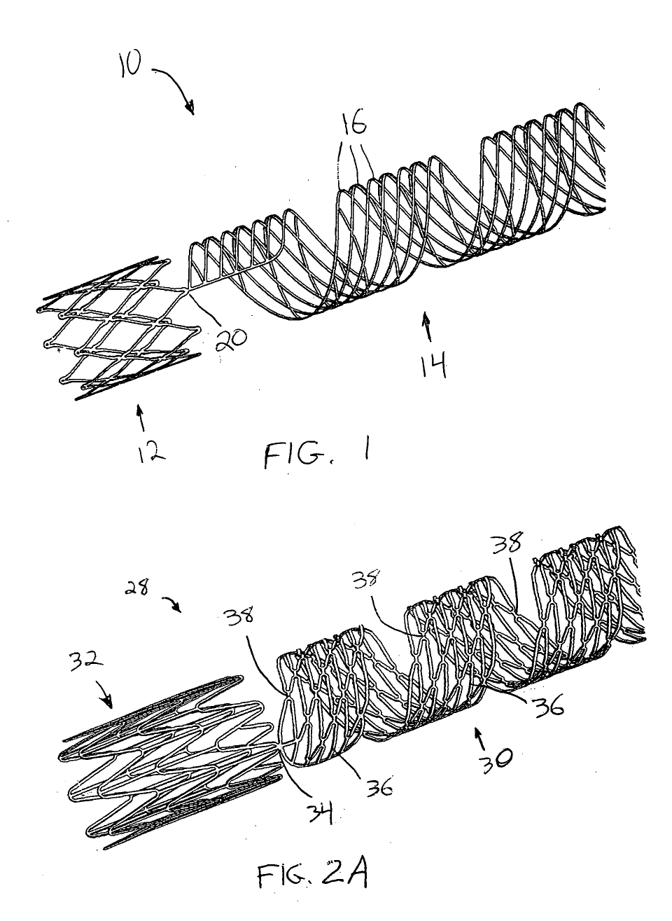

Referring to FIG. 1, vascular prosthesis 10 having improved flexibility according to the principles of the present invention is described. Vascular prosthesis 10 comprises helical body portion 14 and optional distal portion 12, each capable of assuming contracted and deployed states. In FIG. 1, helical body portion 14 and distal portion 12 are depicted in their deployed states. Helical body portion 14 is coupled to distal portion 12 at junction 20.

Vascular prosthesis 10 preferably is formed from a solid tubular member comprising a shape memory material, such as nickel-titanium alloy (commonly known in the art as Nitinol). The solid tubular member then is laser cut, using techniques that are per se known in the art, to a desired deployed configuration, as depicted in FIG. 1. An appropriate heat treatment, also known in the art, then may be applied to the vascular prosthesis while the device is held in a desired deployed configuration (e.g., on a mandrel). Treatment of the shape memory material allows vascular prosthesis 10 to self-deploy to the desired deployed configuration for purposes described hereinafter.

Still referring to FIG. 1, distal portion 12 is designed to be deployed from a stent delivery catheter first to fix the distal end of the stent at a desired known location within a vessel. Helical body portion 14 then may be deployed with great accuracy. In accordance with the principles of the present invention, helical body portion 14 comprises a cell configuration wherein a component of the cells, such as one or more struts 16, extends helically along the helical body portion without hinge points or angular changes. In general, struts that extend substantially parallel to the central axis of the stent provide greater stiffness in the reduced delivery configuration than do helical struts.

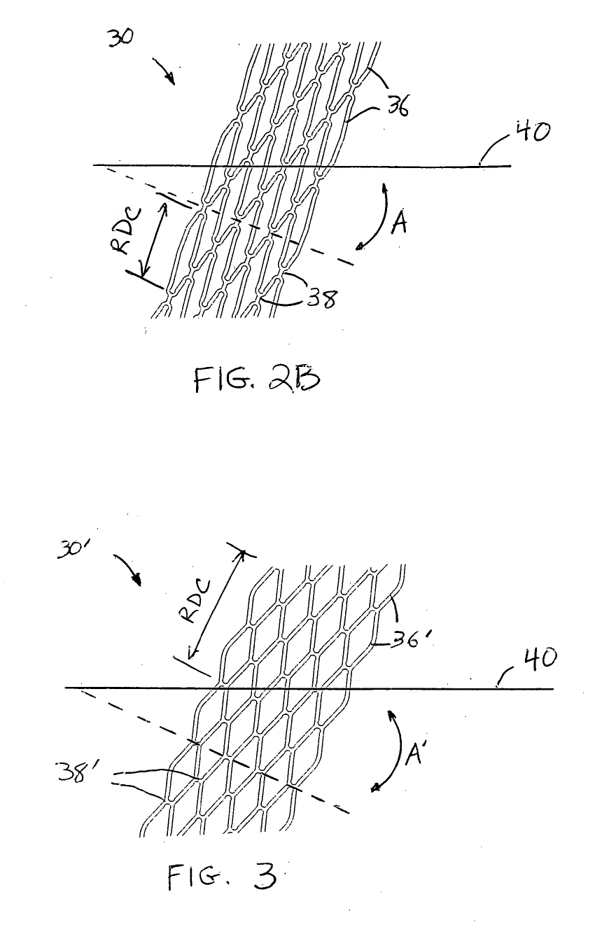

Referring now to FIGS. 2A and 2B, an alternative embodiment of the vascular prosthesis of the present invention is described. Vascular prosthesis 28 comprises helical body portion 30 and optional distal portion 32 coupled at junction 34. Helical body portion 30 comprises a series of cells 36 that are interconnected by hinges 38, wherein each hinge permits articulation of the stent. As depicted in FIG. 2B, cells 36 are substantially diamond-shaped, and aligned substantially end-to-end in a pattern that extends helically for the length of the stent. Adjacent cells are staggered, so that a line extending through the hinges of adjacent cells forms and angle A relative to the longitudinal axis 40 of the stent. It should be understood that cells 36 may have numerous other shapes (e.g., triangular, rhomboidal, pentagonal, etc.) without departing from the scope of the present invention.

In accordance with the principles of the present invention, vascular prosthesis 28 is provided with a high level of flexibility in the both the rolled for delivery state and deployed state by including more hinge points along the helically extending axis of the cells. Alternatively, a high degree of flexibility may be achieved by providing a higher level of angularity along the cell axis. As a further alternative, a combination of increased angularity and hinge points may be employed to achieve a desired degree of flexibility. By adding hinge points 38 and/or alternating the angularity of the struts (e.g., by adding curves or bends), the stiffness of the body portion may be lowered in the reduced delivery configuration. It is therefore possible to control the flexibility of the vascular prosthesis through appropriate hinge placement and angularity. Further, the cells may be used to form patterns of variable metal concentrations and/or radial force values. The ability to vary the metal concentrations may be particularly advantageous in drug delivery applications, e.g., where the vascular prosthesis includes a coating of a bioactive substance, such as a drug that prevents restenosis.

The helical shape of the body portion inherently allows for a greater percentage of metal or scaffolding to be contained per unit length of stent. In accordance with the principles of the present invention, the vascular prosthesis comprises a helical body having a high degree of metal per unit length that retains high flexibility in the reduced delivery profile.

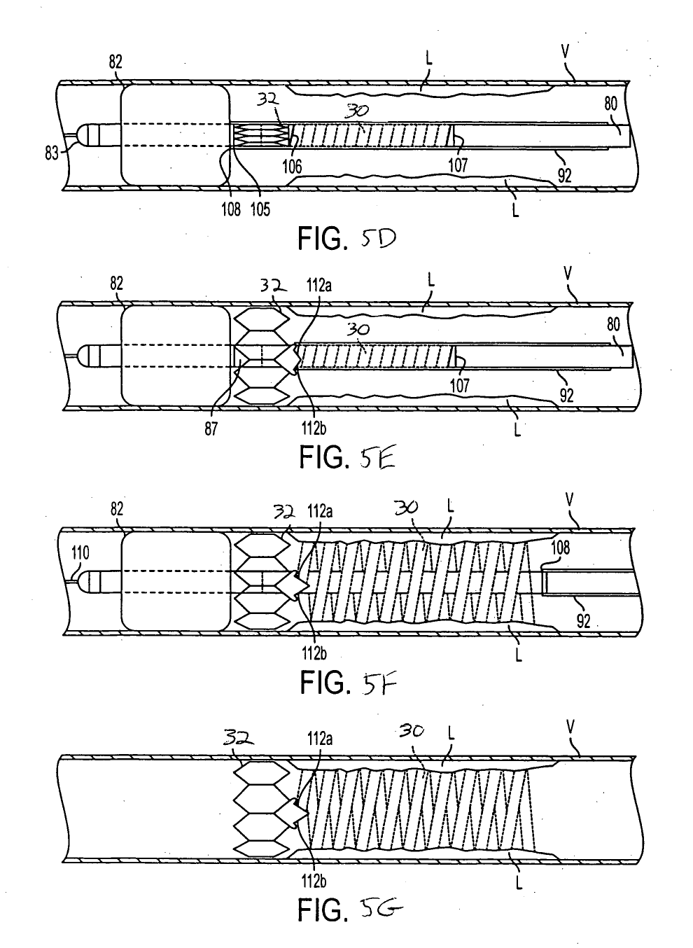

Generally, as the helical body is wound down to a reduced diameter delivery profile (see FIG. 5), each successive wind overlaps the previous wind, thereby causing the stent to become stiffer. Stent flexibility in the reduced diameter delivery profile may be increased by imparting a pattern or cell configuration for the body portion that allows the rolled stent to articulate with limited friction or resistance.

FIGS. 2B shows body portion 30 of the vascular prosthesis of FIG. 2A in an unwound, flattened configuration. Body portion 30 comprises a plurality of cells 36 interconnected by hinges 38 to permit increased flexibility. More particularly, in a preferred implementation, the corners of the cells are coupled by hinges 38 that allow for shape changes to occur in a planar fashion. To enhance flexibility of the stent in the reduced delivery profile, hinges 38 are aligned at an angle A relative to longitudinal axis 40 of the stent. As further depicted in FIG. 2B, the cells have a rounded or elongated diamond shape.

Referring now to FIG. 3, alternative body portion 30′ comprises cells 36′ that are diamond-shaped, but are not rounded or elongated like the cells of the embodiment of FIGS. 2. As in the preceding embodiment, body portion 30′ comprises staggered arrays of cells that extend helically for the length of the helical body portion. In addition, hinges 38 that couple cells 36 together also are aligned at an angle A′ relative to longitudinal axis 40′ of the stent.

Conventional vascular prostheses have a tendency to overlap and tangle when rolled to a reduced delivery configuration. In accordance with one aspect of the present invention, the cells of the vascular prostheses of the present invention are arranged so as to not interengage when rolled to the reduced delivery configuration. It is therefore important to design the cells so that the edge of a cell does not engage the cells on adjacent winds when the vascular prosthesis is rolled for deployment. This is achieved by controlling the edge of the body portion as well as the width of the cells. Specifically, the more linear the edge of the body portion, the less likely the possibility of engagement with other cells. In other words, longer, narrower cells (e.g., as depicted in FIG. 2B) are less likely to become engaged with an adjacent layer of cells that would shorter, wider cells.

As discussed hereinabove with respect to FIG. 2B, hinges 38 of cells 36 preferably are aligned at an angle A with respect to longitudinal axis 40, preferably an oblique angle. The closer the hinges 38 are to being aligned perpendicular to longitudinal axis 40, the stiffer the helical body portion will be in the reduced delivery profile.

The vascular prostheses of the present invention preferably include a “Reduced Delivery Circumference” (RDC), corresponding to the circumference of the vascular prosthesis in the reduced delivery profile. In accordance with another aspect of the present invention, the body portion features a cell pattern based on the RDC. More particularly, the cells that make up the cell pattern are dimensioned so that the length of a cell is an integral fraction of the RDC. Advantageously, a cell pattern that is dimensioned to provide a whole number of cells per RDC allows nesting of the cells along the axis of the vascular prosthesis in the reduced delivery profile. For example, referring to FIG. 2B, body portion 30 features a cell pattern comprising one cell per RDC. In FIG. 3, body portion 30′ features a cell pattern comprising two cells per RDC. As would be appreciated by those of ordinary skill in the art, any whole number of cells per RDC may be employed in the cell pattern without departing from the scope of the present invention.

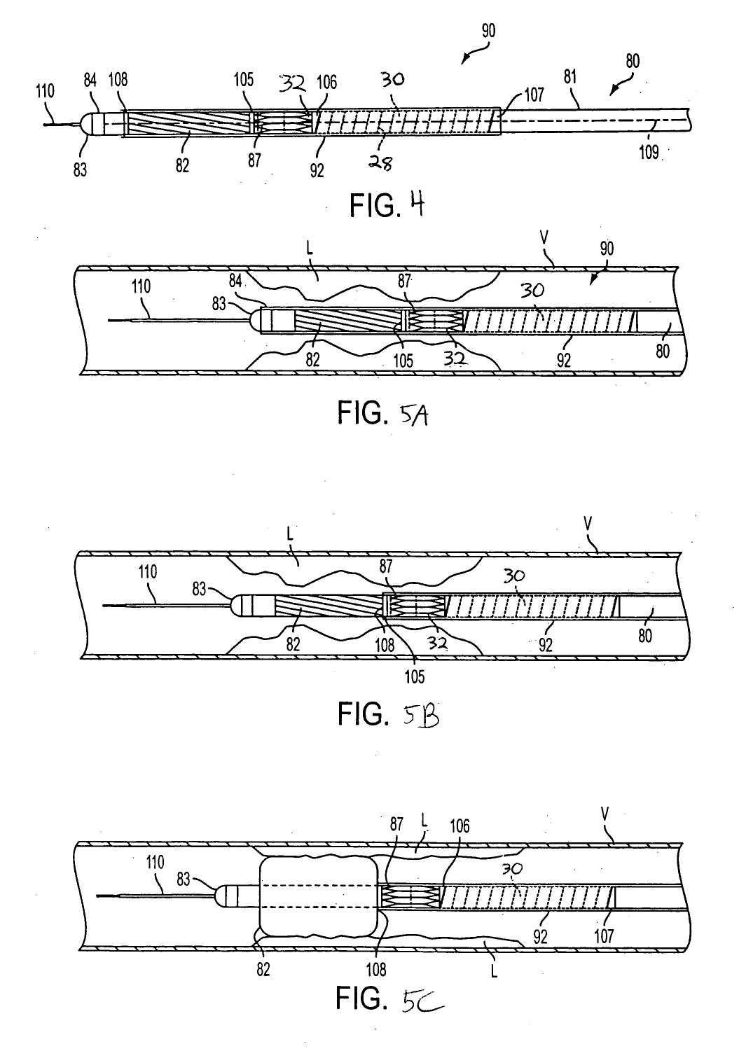

Referring now to FIG. 4, an illustrative delivery catheter suitable for deploying the vascular prosthesis of the present invention is described. In FIG. 4, inner member 80 of the delivery catheter is depicted carrying the inventive vascular prosthesis constrained on inner member 80 by retractable sheath 92. Inner member 81 includes polymer layer 87 that engages the distal end of the distal portion of vascular prosthesis 28 to prevent it from moving proximally when sheath 92 is retracted. Polymer layer 87 preferably is treated, e.g., by formulation, mechanical abrasion, chemically or by heat treatment, to make the polymer tacky or otherwise enhance the grip of the material. Polymer layer 87 may comprise a proximal shoulder of balloon 82, or alternatively may be formed and applied separately from balloon 82. As a yet further alternative, balloon 82 may be omitted, and polymer layer 87 may be disposed adjacent the distal end of the inner member.

Delivery catheter 90 is pre-loaded with vascular prosthesis 28 of the type shown in FIG. 2A, wherein the prosthesis is constrained between inner member 81 and sheath 92. Prosthesis 28 includes distal portion 32 that is engaged with polymer layer 87, and helical body portion 30 that is wrapped to a small diameter around the shaft of inner member 81. Sheath 92 restrains vascular prosthesis 28 against the shaft of inner member 81 until the sheath is retracted proximally. Balloon 82 is shown deflated and wrapped around the shaft of the inner member, in accordance with known techniques.

Sheath 92 is depicted in its insertion configuration, wherein the sheath extends over balloon 82 to a position just proximal of distal end 83. Delivery catheter 90 optionally may include radio-opaque marker bands 105, 106 and 107 disposed, respectively, on inner member 81 beneath the distal and proximal ends of distal portion 32 and at the proximal end of body portion 30. Sheath 92 also may include radio-opaque marker 108 disposed adjacent to its distal end. Delivery catheter 90 preferably includes guide wire lumen 109 that enables the delivery catheter to be slidably translated along guide wire 110.

In operation, delivery catheter 90 is advanced along a guide wire into a vessel containing a treatment area, e.g., plaque or a lesion. Positioning of the vascular prosthesis relative to the treatment area is confirmed using radio-opaque markers 84 and 105-107. Once the delivery catheter is placed in the desired location, sheath 92 is retracted proximally to permit vascular prosthesis 100 to deploy. Polymer layer 87 grips distal portion 32 of stent 28, and prevents distal portion 32 from being dragged proximally into engagement with helical body portion 30 during retraction of sheath 92. Instead, polymer section 87 grips distal portion 32 against axial movement, and permits the distal portion to expand radially outward into engagement with the vessel wall once the outer sheath is retracted.

In addition, as described with respect to FIGS. 5 hereinbelow, either before or after distal portion 32 is expanded into engagement with the vessel wall, balloon 82 is expanded to contact the vessel wall. Balloon 82 therefore anchors distal end 83 of delivery catheter 90 relative to the vessel wall, so that no inadvertent axial displacement of the delivery catheter arises during proximal retraction of the sheath to release distal portion 32 or helical body portion 30 of the vascular prosthesis 28.

Referring now to FIGS. 5, a method of using delivery catheter 90 of FIG. 4 to perform angioplasty and deliver vascular prosthesis 28 of the present invention are described. Vascular prosthesis 28 is disposed in its delivery configuration with distal portion 32 compressed around inner member 80 and retained by sheath 92. Distal portion 32 of prosthesis 28 is disposed in contact with polymer layer 87 to prevent relative axial movement therebetween, as described above.

As shown in FIG. 5A, delivery catheter 90 is percutaneously and transluminally advanced along guide wire 110 until tip 83 of the catheter is disposed within lesion L within body vessel V, for example, as determined by fluoroscopic imaging. Once balloon 82 is positioned adjacent lesion L, sheath 92 is retracted proximally until radio-opaque marker 108 on sheath 92 is aligned with marker 105 of inner member 80, thereby indicating that the sheath has been retracted clear of balloon 82, as shown in FIG. 5B.

With respect to FIG. 5C, once balloon 82 is positioned adjacent lesion L, the balloon may be inflated to dilate a portion of the vessel and disrupt the plaque comprising lesion L. Balloon 82 then may be deflated, moved to another location within the lesion, and re-inflated to disrupt another portion of lesion L. This process is repeated until the lesion has been sufficiently disrupted to restore patency to the vessel.

Referring to FIG. 5D, after performing angioplasty, delivery catheter 90 is advanced so that balloon 82 is disposed adjacent healthy tissue, distal of the lesion. Balloon 82 then is inflated to engage the vessel wall and prevent axial displacement of the delivery catheter during subsequent retraction of sheath 92. Polymer layer 87 engages distal portion 32 of vascular prosthesis 28, thereby preventing axial displacement of distal portion 32 during retraction of sheath 92.

With respect to FIG. 5E, after balloon 82 is inflated to engage the vessel wall, sheath 92 is retracted proximally until distal portion 32 self-expands into engagement with vessel wall within or distal to lesion L. Proximal movement of sheath 92 may be halted once radio-opaque marker 108 of sheath 92 is substantially aligned with radiopaque marker 106 of inner member 80. When released from the constraint provided by sheath 92, the struts of distal portion 32 expand in a radial direction to engage the interior of vessel V. Stress relieving articulation, comprising connection members 112a and 112b, permit distal portion 32 to engage into engagement with the wall of vessel V while mitigating torsional forces applied to the distal edge of helical body portion 30.

Referring now to FIG. 5F, after distal portion 32 is secured to the vessel wall distal of lesion L, sheath 92 is further retracted proximally to cause the helical body portion of stent 28 to unwind and deploy to its predetermined shape within vessel V. During proximal retraction of sheath 92, each subsequent turn unwinds one at a time and engages and conforms to an inner wall of vessel V in a controlled manner.

Torsional forces applied to distal portion 32 during retraction of sheath 92 are uniformly distributed over the surface of balloon 82, thereby reducing the risk of insult to the vessel endothelium. Once the last turn of the helical body portion of stent 28 is deployed, balloon 82 is deflated, and the sheath optionally may be advanced to cover balloon 82. Delivery catheter 90 then is withdrawn from the patient's vessel, and guide wire 110 is removed, completing the procedure.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

1. An implantable vascular prosthesis having improved flexibility, comprising:
a helical body portion having a length, a reduced delivery configuration and an expanded deployed configuration, the helical body portion comprising a plurality of cells aligned helically along the length of the helical body portion to enhance flexibility of the vascular prosthesis in the reduced delivery configuration.

2. The vascular prosthesis of claim 1, wherein the plurality of cells comprises one or more substantially parallel struts.

3. The vascular prosthesis of claim 1, wherein the plurality of cells is interconnected by hinges and the cells are aligned substantially end-to-end along the length of the helical body portion.

4. The vascular prosthesis of claim 3, wherein the plurality of cells are diamond-shaped, triangular, rhomboidal, or pentagonal.

5. The vascular prosthesis of claim 3, wherein each cell includes a plurality of corners and the hinges are disposed at the corners to allow shape changes to occur in a planar fashion.

6. The vascular prosthesis of claim 1, wherein plurality of cells is arranged in rows that are staggered relative to a longitudinal axis of the vascular prosthesis.

7. The vascular prosthesis of claim 1, wherein a radial force of the vascular prosthesis is controlled by varying placement of the hinges.

8. The vascular prosthesis of claim 6, wherein a radial force of the vascular prosthesis is controlled by varying an angle at which adjacent rows are staggered.

9. The vascular prosthesis of claim 1, wherein the plurality of cells is configured to reduce interengagement of turns of the helical body portion in the reduced delivery configuration.

10. The vascular prosthesis of claim 9, wherein the cells are elongated along the length of the helical body portion.

11. The vascular prosthesis of claim 1, wherein the hinges of adjacent rows of cells are aligned at an angle with respect to a longitudinal axis of the vascular prosthesis.

12. The vascular prosthesis of claim 11, wherein a radial force of the body portion is controlled by varying the angle at which the hinges of adjacent rows of cells are aligned.

13. The vascular prosthesis of claim 1, wherein the helical body portion has a reduced diameter circumference representing a circumference of the helical body portion in the reduced delivery configuration.

14. The vascular prosthesis of claim 13, wherein each cell of the plurality of cells is sized so that cell length is an integral fraction of the reduced diameter circumference.

15. The vascular prosthesis of claim 14, wherein each cell of the plurality of cells is sized so that cell length is an integral number of cells per reduced diameter circumference.

16. The vascular prosthesis of claim 1 wherein the helical body portion has a distal end, the vascular prosthesis further comprising a radially self-expanding tubular anchor coupled to the distal end of the helical body portion.

17. The vascular prosthesis of claim 1 further comprising a coating that elutes a bioactive substance.

18. A stent comprising a helical portion having a length, a delivery configuration and a deployed configuration, the helical portion comprising a plurality of cells aligned end-to-end helically along the length of the helical portion.

19. The stent of claim 18, wherein the plurality of cells is interconnected by hinges that are aligned along the helix of the helical portion.

20. The stent of claim 18, wherein the plurality of cells are diamond-shaped, triangular, rhomboidal or pentagonal.

21. The stent of claim 18, wherein each cell includes a plurality of corners and the hinges are disposed at the corners to allow shape changes to occur in a planar fashion.

22. The stent of claim 18, wherein plurality of cells is arranged in rows that are staggered relative to a longitudinal axis of the vascular prosthesis.

23. The stent of claim 18, wherein the plurality of cells is configured to reduce interengagement of turns of the helical portion in the delivery configuration.

24. The stent of claim 18, wherein the cells are elongated along the length of the helical body portion.

25. The stent of claim 18, wherein the hinges of adjacent rows of cells are aligned at an angle with respect to a longitudinal axis of the vascular prosthesis.

26. The stent of claim 18, wherein the helical portion has a reduced diameter circumference representing a circumference of the helical portion in the delivery configuration, each cell of the plurality of cells sized so that cell length is an integral fraction of the reduced diameter circumference.

27. The stent of claim 18 wherein the helical portion has a distal end, the stent further comprising a radially self-expanding tubular anchor coupled to the distal end of the helical portion.

28. The vascular prosthesis of claim 18 further comprising a coating that elutes a bioactive substance.
